(19) United States
(12) Patent Application Publication
(10) Pub. No.: US 2009/0166562 A1
(43) Pub. Date: Jul. 2, 2009

(54) Production of microfluidic devices using laser-induced shockwaves

(76) Inventor: Micah James Atkin, Glen Huntly (AU)

Correspondence Address: CLARK & ELBING LLP, 101 FEDERAL STREET, BOSTON, MA, 02110, US

(21) Appl. No.: 12/308,019

(22) PCT Filed: Jun. 7, 2007
(86) PCT No.: PCT/AU2007/000802

§ 371 Date: Mar. 12, 2009

Related U.S. Application Data
(60) Provisional application No. 60/811,437, filed on Jun. 7, 2006.

(30) Foreign Application Priority Data
Jun. 7, 2006 (AU) 2006903098
Nov. 22, 2006 (IB) PCT/IB2006/003311
Jan. 11, 2007 (AU) PCT/AU2007/000012
Jan. 24, 2007 (AU) PCT/AU2007/000061
Jan. 24, 2007 (AU) PCT/AU2007/000062
Apr. 10, 2007 (AU) PCT/AU2007/000435

(51) Int. Cl. A61N 5/00

(52) U.S. Cl. 250/492.1

(57) ABSTRACT

A method an apparatus for manufacturing a microfluidic device (10) is disclosed in which a laser is used to remove selected portions of one of the layers that make up the device. The portion of the layer may be removed before the layer is amalgamated with other layers making up the device, or the portion may be removed after the layers have been bonded together. The laser beam used to accomplish removal is a combination of at least two laser beams (3, 4), one of which (3) may be a continuous beam to form a melt of the portion to be removed, the other (4) being pulsed or modulated in some way to periodically induce shockwaves which remove the portion. The laser beams use at least one part (5, 8, 9) of the same alignment system.

### CROSS REFERENCE TO RELATED APPLICATIONS

This application claims priority from U.S. provisional patent application No. 60/811,437, filed on 7 Jun. 2006 the entire contents of which are incorporated herein by reference. This application also claims priority from Australian provisional patent application AU 2006903098 filed on 7 Jun. 2006, the entire contents of which are incorporated herein by reference.

This application also claims priority from International (PCT) application PCT/IB2006/003311, filed on 22 Nov. 2006, the entire contents of which are incorporated herein by reference. This application also claims priority from International (PCT) application PCT/AU2007/000012, filed on 11 Jan. 2007, the entire contents of which are incorporated herein by reference. This application also claims priority from International (PCT) application PCT/AU2007/000061, filed on 24 Jan. 2007, the entire contents of which are incorporated herein by reference. This application also claims priority from International (PCT) application PCT/AU2007/000062, filed on 24 Jan. 2007, the entire contents of which are incorporated herein by reference. This application also claims priority from International (PCT) application PCT/AU2007/000435, filed on 10 Apr. 2007, the entire contents of which are incorporated herein by reference.

### FIELD OF THE INVENTION

This invention relates generally to manufacturing methods and devices for laser machining single or multilayer materials. The field of this invention also extends to the manufacture of components relating to food and pharmaceutical, medical, in vitro diagnostic, and microfluidic devices and packaging.

### BACKGROUND OF THE INVENTION

The present invention relates generally to manufacturing methods and devices for laser machining materials. Typically laser processing of devices has been in the areas of laser cutting, surface machining, surface treatment, and laser welding. Laser cutting typically involves cutting entirely through a substrate; surface machining techniques selectively remove parts of a substrate; physical surface treatment involves melting or etching the surface, whereas chemical surface treatment typically operates below the ablation threshold to modify the surface properties; and laser welding typically involves selectively melting the interfacial material between two surfaces, and can be performed by either direct surface exposure, or through the use of transmission or reverse conduction welding for joining internal surfaces. Scanned beam systems are known for all methods and lithographic systems have been used for structuring and surface modification depending on the energy density, material properties, resolution, and throughput required.

Applications for the laser processing of multilayer materials typically involve the removal of outer layers of material, such as the stripping of insulation off wires or exposing electrodes on printed circuit boards, or welding via transmission and reverse conduction methods.

Transmission laser welding operates by one material being transparent to and the other material being an absorber of the irradiated laser wavelength. This allows the laser beam to selectively heat between the two materials producing localised welding when the heat rises above the glass transition temperature. For integration into the production environment, the main limitations are processing times, and limitation of compatible materials and number of layers that can be processed.

Reverse conduction welding operates in a similar manner to transmission layer welding except that the heat is generated by laser absorption at a backplane. The polymer films clamped above the absorbing layer conduct the heat from its surface and locally melt. Due to uniform heat conduction within the polymers which limits spatial resolution, the technique is only suitable for thin films and relatively large structures.

More recently specific laser absorbers, such as Clearweld®, have been used for bonding. In practice this material is difficult to apply to mass production of micromachined substrates and produces a slightly opaque weld that can reduce the appeal of a product or interfere with the operation, for example, sensor response, of some devices.

Lasers have also been used for micromachining substrate surfaces. These techniques usually employ ultraviolet (UV) lasers, typically excimer lasers, which can produce fine anisotropically etched structures down to one micron. Unfortunately such systems are expensive and relatively slow to process material. More recently, focus has been on the use of shorter wavelength UV lasers that can machine channels down to 100 μm, depending on the material thickness. Unfortunately such systems provide a large heat-affected zone that limits fine structures, such as those required for microfluidic geometries. In a similar manner, infrared (IR) YAG and CO₂ lasers have been demonstrated for microfluidic channel fabrication for large structures only (in the order of hundreds of microns).

The challenge in incorporating such technologies into manufacturing processes relates to the time required for the laser to complete its machining process as well as the quality morphology of the resulting cut or machined surface.

The reference to any prior art in this specification is not, and should not be taken as, an acknowledgement or any form of suggestion that the prior art forms part of the common general knowledge.

### SUMMARY OF THE INVENTION

The invention provides methods for laser structuring of single and multilayered materials. The invention includes apparatus, methods and products.

The method, apparatus and devices of the present invention have many advantages, including in various embodiments, for example:
a smaller heat affected zone
improved structure feature size
improved structuring accuracy
improved structuring precision
simplified fabrication of parts with unsupported structures
use of cheaper lasers such as IR YAG and CO₂ lasers for microstructuring
parallel processing

In a first aspect of the invention, there is provided a method for manufacturing at least part of a device comprising a substrate wherein at least one laser is used to alter a portion of the substrate during the manufacturing process. Certain embodiments provide such a method for manufacturing at least part of a multilayered device comprising use of at least one laser to alter at least one layer of said part during the manufacturing process.

In a second aspect of the invention, there is provided, an apparatus for manufacturing at least part of a device comprising a substrate, the apparatus comprising at least one laser source to produce a laser beam to alter at least one portion of the substrate during the manufacturing process. Certain embodiments provide such an apparatus for manufacturing at least part of a multilayered device comprising at least one laser source to produce a laser beam to alter at least one layer of said part during the manufacturing process.

In a third aspect of the invention, there is provided a part of a device manufactured according to the process or using the apparatus of the present invention.

In a fourth aspect of the invention, there is provided a device manufactured according to the process or using the apparatus of the present invention.

Some preferred embodiments are particularly adapted to manufacture of specialist devices, such as microfluidic devices.

Any suitable laser with any suitable characteristics may be used in the method or apparatus of the present invention. For example, in some embodiments, a variety of wavelengths are utilized and in others, a plurality of laser beams.

In embodiments which comprise a plurality of laser beams, the laser beams may for example improve the formed structure and/or simplify the manufacturing process. In some embodiments, the plurality of laser beams use at least one part of the same alignment system. The plurality of laser beams may interact with each other or be used in ways which enhance the overall processing. Thus, for example, the plurality of laser beams may operate at least partially simultaneously or they may operate optionally at least partially concurrently or at least partially intermittently. The plurality of laser beams may also be operated with one or more timing characteristics.

In some embodiments, the laser beam energy is increased which may for example enable faster processing. Thus in some embodiments, the increased laser beam energy enables alteration of the dominant processing mechanism, which is optionally one or more of thermal melt, plasma formation, ablation by bond cleavage and subsequent volume expansion, and multi-photon bond dissociation.

Embodiments with a plurality of laser beams may also enable simplified manufacturing processing, for example by reducing cost, improving alignment, increased speed of processing, and optionally for example when a plurality of beams use parts of the same alignment system.

In some embodiments, a first laser beam and a second laser beam work in conjunction with one another. In one such embodiment, a first laser beam forms a melt and a second laser beam removes material, optionally by laser induced shockwaves and optionally by a pulsed laser beam. In another embodiment, a first laser beam increases bond or lattice energy to an excited state and a second laser beam removes material, optionally with an increased energy density. In a further embodiment, a first laser beam removes material and a second laser beam alters surface morphology, optionally by inducing surface reflow for reshaping, debris minimisation, crystallinity changes, and/or surface chemistry alteration. In some embodiments a first laser beam having a first wavelength is used to target a first portion of substrate and a second laser beam having a second wavelength is used to target a second portion of substrate. In some of these embodiments applicable to multilayered devices, the first laser targets a first layer and the second laser targets a second layer. In other embodiments, the first laser beam targets a particular chemical bond in the substrate and a second laser beam having a second wavelength is used to target a different chemical bond in the substrate.

In some embodiments which comprise a plurality of laser beams, the beams may be combined prior to falling incident on a portion of substrate or a layer. Combination of the beams may be by any suitable method, for example, by using an optical element, such as a mirror or lens. In some embodiments, the plurality of laser beams originally arise from the same source.

The material to be lasered may be of any suitable form. Some preferred embodiments comprise the use of an additive in a layer to alter the effect of a laser beam on that or another layer. Thus, for example, the additive may affect and optionally improve radiation absorption at the laser's wavelength. Equally, however, it may increase transmission of a laser beam through the substrate and consequently indirectly affect the substrate or layer below. Some embodiments comprise the use of a portion of substrate (which may for example, be a layer) with an absorption and/or reflection characteristic to influence the effect of the laser. The characteristic may be of any suitable form, for example, it may allow selective machining of an absorbing portion of substrate (which may for example, be a layer).

Other suitable aspects of the material to be lasered may be provided, altered, or optimised. For example, the material may comprise a thermally conductive portion (which may for example, be a layer) for improved structure formation.

Various thermal techniques may also be used as part of the present invention. For example, heat may be reduced or guided to provide improved structure geometry or reduce the effect of the machining process on the surrounding materials and structures.

Various masking techniques may also be used as part of the present invention. Thus, one embodiment comprises the use of a masking component between the laser source and a portion of substrate (such as a layer) to limit or alter exposure to the laser beam on an area of the substrate or layer. The mask or masking component may take any suitable form, for example, in applications relating to multi-layer devices, the masking component may itself be a portion of the substrate or a layer.

The present invention may also be used to increase throughput, for example by providing parallel processing. In some such embodiments a masking component may contribute to alignment of parts during manufacture. In some embodiments a masking component provides greater spatial resolution. The masking component may perform one or more functions, such as for example: conducting heat away from an area on a portion of substrate, such as a layer, (b) protecting a surface from debris, and/or (c) supporting one or more structures during processing.

The present invention may be further optimised with the use of an optical component to alter or focus the laser beam. The optical component may take any suitable form, for example it may comprise one or more lenses, prisms or other refractive, diffractive or reflective elements. In some embodiments, the optical component simplifies alignment of parts during processing. The optical component may perform one or more functions such as for example, altering one or more of the frequency, intensity, direction, duration or timing of the laser beam.

In some embodiments of the present invention, a portion of substrate such as a layer may be removed during or after the manufacturing process. The use of such a removable portion of substrate or layer, in some situations referred to as a sacrificial portion or sacrificial layer, can add further benefits to the present invention. In some embodiments which comprise such a portion of substrate or layer which is removed, the removed portion may perform one or more of the following functions: protect a surface from debris, thermal conduction, support cut out or free standing structures, focus or mask a beam, allow a secondary machining process to occur.

The substrate material and/or layers the subject of the laser processing and/or manufacturing of the present invention may be of any suitable type. Thus, for example, they may comprise one or more of polymer, metal, metal oxide, metal foil, paper, nitrocellulose, glass, silicone, photo-resist, ceramic, wood or fabric.

The process flow of a method and apparatus according to the present invention may be arranged in any suitable manner. In some embodiments, the process utilizes an at least semi-continuous web while in others, the process is not web-based.

The method and apparatus of the present invention is also particularly suited to the use of additional non-laser processing steps which may occur before, during or after a laser step. Any suitable non-laser step may be used in conjunction with the present invention. Thus, in some embodiments, a non-laser process step comprises one or more of injection molding, micromilling, die cutting, hot foil stamping, stamping, embossing, thermoforming, print-head deposition, photolithography, coating, curing. In some embodiments, a non-laser processing step comprises a pre-treatment process, which may for example reduce the heat affected zone from the laser machining process. A pre-treatment process according to the present invention may comprise any suitable steps, thus for example, it may comprise one or more of: providing cooling or heat sinking to parts of the material, or modifying the material's surface or bulk properties to alter the thermal conductivity or absorption characteristics.

In some embodiments, there is further provided a post-treatment process which may for example optionally structure, cure, surface treat, coat or render one or more parts.

The application of thermal energy, or heat is one example of a non-laser processing step which may have particular benefits. In one embodiment, one or more of the area of the substrate or layer to be laser treated, the local area on the substrate or a tool may be heated to improve material flow around a tool. Any suitable tool may be used, for example, it may be an embossing tool. In one embodiment, a laser beam is scanned over an area to be embossed. Such scanning may occur at any suitable timed, for example prior to, during or after embossing.

In some embodiments, a structure is formed by selectively applying a laser to a defined area of a substrate or layer to thereby weaken it. Such a process step may be used to make a wide variety of useful structures, for example, burst valves, tearing guides, perforations, meshes, etc. Some embodiments utilise the laser to alter the barrier properties of a portion of substrate or layer by selective application of the laser. This may occur by any suitable means, for example a series or network of perforations through a portion of substrate or layer.

A laser treatment step according to the present invention may occur at any suitable stage. For example, a component part of a device to be manufactured in accordance with the invention may be laser treated prior to or after assembly of the device. In some embodiments, assembly of a multilayered device comprises laser treatment. This may occur for example where assembly comprises a laser-treatment bonding step which may for example comprise laser assisted bonding of layers.

Precision alignment is a very important part of certain embodiments of the present invention. In some embodiments, the method or apparatus comprises the use of one or more alignment marks, notches, grooves, or edge guides for alignment. Some embodiments also comprise the use of a control system. Any suitable control system may be used, for example it may comprise one or more of: mechanical sensor feedback, optical sensor feedback, part translation and/or laser scanning adjustment.

Throughout this specification (including any claims which follow), unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

### BRIEF DESCRIPTION OF DRAWINGS

FIGS. 1A and 1B are schematic representations of examples of combinations of multiple laser beams.

FIG. 2 is a schematic representation of a card or sheet production system.

FIG. 3 is a schematic representation of a web or continuous production system.

FIG. 4 is a schematic representation of a combined laser and embossing process.

FIG. 5 is a schematic representation of a simultaneous laser and embossing process.

FIG. 6 is a schematic representation of laser structuring in multilayer devices with and without heat conductive layers.

FIG. 7 is a schematic representation of selective laser machining of layers in a multilayer device.

FIG. 8 is a schematic representation of the use of reflective lasers during laser machining in a multilayer device.

FIG. 9 is a schematic representation of examples of microfluidic fabrication by laser machining.

FIG. 10 is a schematic representation of an example of a microfluidic device fabricated through a transparent layer by laser machining.

FIG. 11 is a schematic representation of the use of masking on a multilayer device for laser processing.

FIG. 12 is a schematic representation of the use of optical components on a multilayer device for laser processing.

FIG. 13 is a schematic representation of the use of protective layers during the laser machining process.

FIG. 14 is a schematic representation of examples of burst valve formation by laser machining.

FIG. 15 is a schematic representation of an example of a tear structure machined into a multilayer device.

FIG. 16 is a schematic representation of an example of modification of a multilayer device for controlled barrier layer properties.

### DETAILED DESCRIPTION OF THE INVENTION

It is convenient to describe the invention herein in relation to particularly preferred embodiments relating to food and pharmaceutical, medical, in vitro diagnostic, and microfluidic devices and packaging. However, the invention is applicable to a wide range of situations and products and it is to be appreciated that other constructions and arrangements are also considered as falling within the scope of the invention. Various modifications, alterations, variations and or additions to the construction and arrangements described herein are also considered as falling within the ambit and scope of the present invention.

As used herein, the term “fluid” refers to either gas or liquid phase materials. As used herein, the term “microfluidic” refers to fluid handling, manipulation, or processing carried out in structures with at least one dimension less than one millimetre. As used herein, the term “beam” or “ray” refers to more than one photon travelling in a substantially similar direction. Laser machining techniques used in the present invention include, but are not limited to, scanned beam and lithographic systems. Laser and material interactions used in the present invention may be of any suitable type, and may for example include photo-thermal, photo-chemical processes or combinations of the two.

The laser beam incident on the substrate or material may be from a single laser or a plurality of lasers. Where multiple laser beams are combined to machine the work-piece, the beams may operate simultaneously or with different timing characteristics. For example laser beams may operate at the same or different wavelengths irradiating the same area either, alternatively, concurrently, or simultaneously at different switching frequencies.

Various improvements are made possible by combining multiple beams, such as for example, increasing beam energy density to provide faster processing. In addition, a combination of multiple beams increases beam energy density which enables alteration of the dominant processing mechanisms, such as thermal melt, plasma formation, ablation by bond cleavage and subsequent volume expansion, and multi-photon bond dissociation. Furthermore, a combination of multiple beams may simplify manufacturing implementation by reducing alignment issues and by increasing the speed of processing when the beams are delivered using the same alignment mechanism. Some examples include: alignment mechanisms may be in the form of the laser beams using separate optical paths and a common alignment controller, or the beams may share a common optical path, such as where the laser beam guiding stage is common to both beams. An example of this would be where galvo mirror scanners or x-y driven output optics are common to both laser beams. Such improvements in manufacturing are particularly important for micro-structuring to avoid the use of additional costly alignment systems, which would also introduce a further tolerance requirement associated with the error in beam placement between the multiple alignment systems. A further advantage of using multiple beams is that it enables the use of multiple processing methodologies which mean faster processing and improved structure formation. This may be done in various ways, for example:
Melt formation from a first laser beam and material removal by laser induced shockwaves from a second laser beam. Examples include the combination of a continuous laser beam for melt formation with a pulsed laser beam to induce material removal.
Using a first laser beam to increase bond energy and a second laser beam to remove material. The first laser beam increases bond or lattice energy to an excited state, but does not increase energy density to the point that the bonds dissociate. The second laser beam, which has greater photon energy is used to induce bond dissociation and therefore removal of material.
Material removal by a first laser beam and surface morphology alteration by a second beam. The second beam may for example induce surface reflow for reshaping, debris minimisation, crystallinity changes, and/or surface chemistry alteration. Either laser beam may use thermally or ablative mechanisms.
A first laser beam induces a material change, such as crystallinity, bond chemistry, or surface morphology, and a second laser beam removes material. For example the first laser beam may increase the absorption characteristics of the material to the second laser beam, or alternatively be used to selectively reduce the absorption characteristics of the material to the second laser beam.
Use of laser beams with different wavelengths to target different processing materials. Thus, for example, different bond or vibrational energies may be targeted in the same material by different wavelengths, or the different lasers may target different materials or layers when multiple materials are processed, as with multilayered devices.

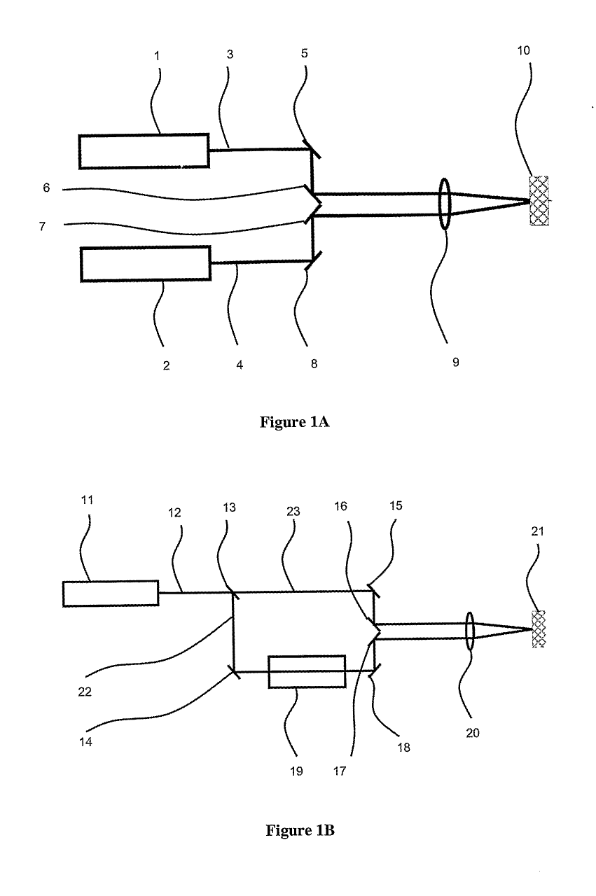

In one embodiment, multiple laser beams are combined prior to irradiating the material; as shown in FIG. 1A in which beams (3, 4) from two separate lasers (1,2) are combined by reflective mirrors (5,6,7,8) and lens system (9) to machine the work-piece (10). FIG. 1B illustrates an example in which a laser beam (12) from a single laser source (11) is split at the partially reflective mirror (13) into two separate beams (22,23) with one beam (22) being altered (in timing or wavelength) by the altering system (19), which could for example be a delay line, switched gate, or frequency multiplier, before being recombined through the mirror elements (15,16,17,18) and lens system (20) to machine the work-piece (21). Altering of the Laser beams may be by any suitable means, for example, (a) frequency, such as a frequency multiplication as for example by a YAG Laser beam that has its fundamental frequency of 1.06 μm quadrupled to 266 nm, or (b) duration, such as a continuous wave laser beam that is switched to a pulsed waveform.

The laser machined structures may be fabricated on discrete parts or onto reels of continuous material. FIG. 2 shows one embodiment of a production line used to structure discrete parts or items such as cards. In this example the laminated material may be stamped in the system prior to lamination or be converted as a separate process. The process depicts input/output hoppers (24,25) and a card handling system that accepts cards (26) in ISO 7816 format material. The processes which are sequentially operating on the cards include: laser machining system (27), overlay laminating (28) of preformed laminates (32), embossing (29), topping (30), and finally programming or encoding (31).

An example of a production line for the fabrication of continuos parts, or onto a web, is illustrated in FIG. 3. In this example, the modular production units depicted are interspersed with material feed handlers (43) and include: forming stock material inputs (33), blister forming (34), filling (35), bonding (36), printing (37), curing (38), tension control (39), material guides and unwinds (40), laser structuring through composite materials (41), die cutting (42), and final part collection (44).

Structures produced according to the present invention may be cut, rendered or divided into smaller parts.

In one embodiment of the invention, laser machined parts are bonded to other components, which may or may not be a continuous substrate, and may or may not be planar, and may be made of single or multiple components.

In another embodiment, the laser machining processes may be combined with other structuring processes; such as injection molding, micromilling, die cutting, hot foil stamping, stamping, embossing, thermoforming, print-head deposition, photolithography, coating, curing and other structuring methods.

The present invention may also be combined with other processes to facilitate the laser machining process or improve the performance of laser machined devices. For example the present invention may be combined with one or more pre-treatment processes to reduce the heat affected zone from the laser machining process. Such pre-treatment may include providing cooling or heat sinking to parts of the material, or modifying the material's surface or bulk properties to alter the thermal conductivity or absorption characteristics. Post-treatment processes may also be used to structure, cure, surface treat, coat or render the parts. For example PCT/AU2007/000061 describes a combined laser embossing process that enables more rapid replication of embossed features than normal and hot embossing. By pretreating the local area to be embossed with lasers, the local material is altered, which allows (a) lowering of the softening point (as is especially the case with orientated films), preheating of the exposed area, (b) material reflow and (c) in some cases, ablation from the embossed area.

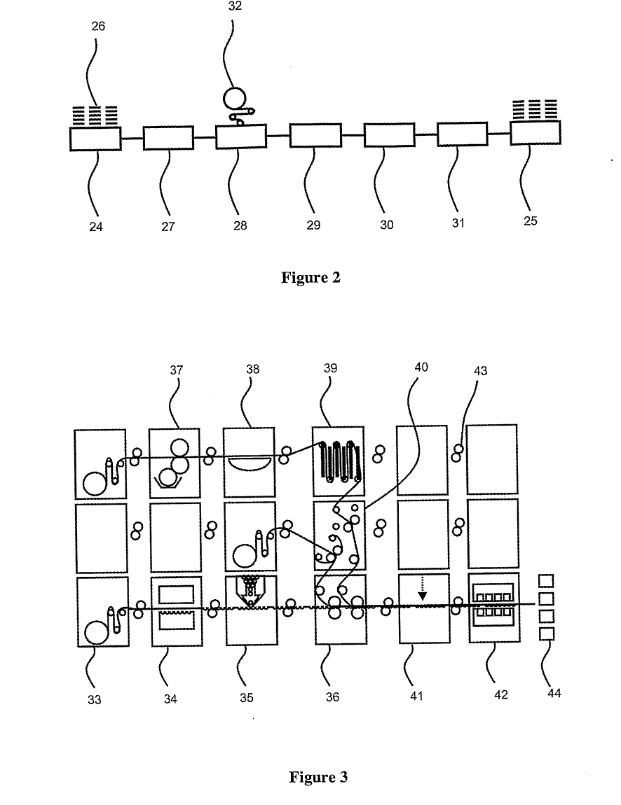

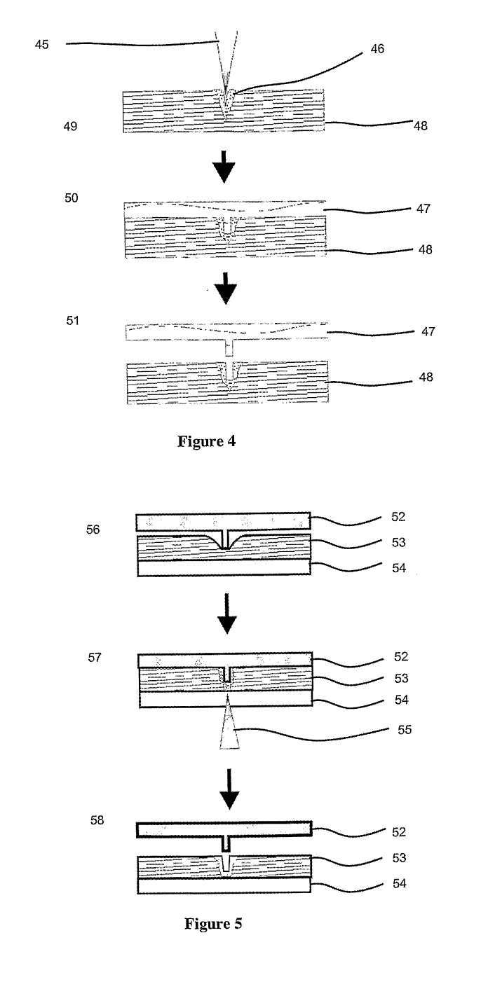

After laser processing, and before stamping, the area of the film to be treated, the local area on the substrate or the tool may be heated to improve the material flow around the tool. The laser beam may expose the entire substrate surface or just the area to be embossed, as illustrated in FIG. 4 in which a focused laser beam (45) is scanned over the embossed area (46) prior to embossing (49). The material in the embossing area (46) then forms around the embossing tool (47) during embossing (50), replicating the tooling structure into the material (48) when the tool (47) is removed (51). Such a process allows the use of longer wavelength lasers than the expensive and slower UV excimer systems for fine structure formation. Unlike their excimer counterparts, such longer wavelength systems produce more thermal damage and typically have larger focus spot sizes, which severely limits their spatial resolution for micro-structuring. By combining the laser machining process with embossing, finer and more accurately formed microstructures may be fabricated than with the laser alone, and larger structures may be formed than with embossing alone. Thereby providing a much faster and cheaper method than excimer laser processing. Similarly, the swarf and rough edges produced by the laser cutting processes may also be processed after structuring to improve channel performance.

The combination of other processes with laser process may occur either simultaneously or in any order. In some embodiments, it occurs simultaneously. For example, in one embodiment an embossed material is laser machined during the embossing processes. Whilst the embossing tool is pressed to the surface of the material, the laser irradiates the reverse side of the material to cause localised reflow around the tool to improve the speed of embossing, and or the replication of the structure from the embossing process. Processing in this manner also helps to relieve some of the induced stresses in the material around the reflowed area, which is critical in microstructure formation where the induced stresses can cause structure deformation when the tool is removed. A material transparent to the lasing wavelength is typically used to support the embossed material during such a process. In an alternative arrangement, the laser absorbing layer may be a thin layer located thermally close to the embossing area, and the substrate may be transparent, so that upon laser irradiation the embossed area is heated by the absorbing layer. FIG. 5 illustrates a tool embossing into a surface prior to irradiation (56), during irradiation (57), and then removal of the tool after irradiation (58). In these steps the material (53) being embossed is supported by a carrier layer (54) which is transparent to the laser beam (55), to enable irradiation of the material (53) whilst it is in contact with the embossing tool (52).

The use of alignment marks, notches, grooves, and or edge guides are common approaches used for alignment in many manufacturing systems. In one preferred embodiment of the process, the present invention uses control systems to facilitate alignment and provide quality control. Parameters in the control system include, but are not limited to, mechanical and/or optical sensor feedback with part translation or laser scanning adjustment for improved alignment.

In certain preferred embodiments of the invention, one or more materials may include the use of specific absorber additives to improve the material's absorption at the laser's wavelength.

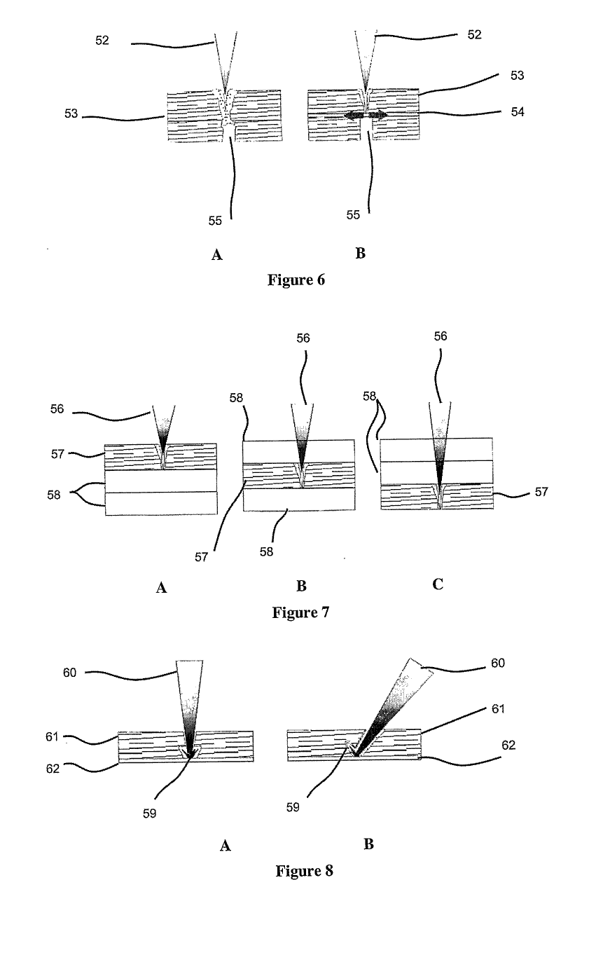

In certain preferred embodiments of the invention the device or component to be laser processed is made of multi-layered materials. One or more layers of the material may have different heat conduction characteristics allowing improved structure formation. For example, FIG. 6 A illustrates laser beam (52) cutting a substrate material (53) with no addition of thermal conductive layers, and FIG. 4 B shows the laser machining of a multilayer substrate with a thermally conductive layer (54) providing heat conduction during the machining process. This technique can be used to reduce and or guide the heat affected areas during the machining process to provide improved structure geometry or reduce the machining processes effect on the surrounding materials and structures (55).

In certain preferred embodiments of the invention the device or component to be laser processed is made of multi-layered materials. One or more of the layers of the material may have different absorption characteristics allowing selective machining of the absorbing layers, as illustrated in FIG. 7. FIGS. 7 A, B, and C show selective machining by the laser beam (56) of the top, middle, and bottom layers, respectively, with different configurations of absorbing (57) and transmission (58) layers.

In certain preferred embodiments of the invention the device or component to be laser processed is made of multi-layered materials. One or more of the layers of the material may have different absorption and or reflection characteristics allowing the selective machining of absorbing layers. As illustrated in FIGS. 8 A and B in which the undercut structures (59) are machined by the laser beam (60) passing through the substrate material (61) and being reflected by surface (62).

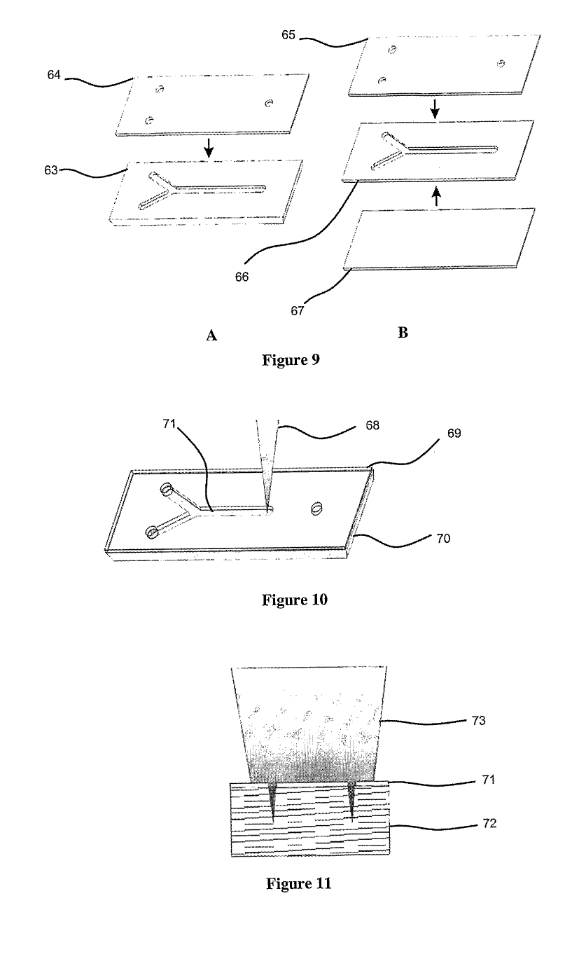

In another preferred embodiment of the invention the multi-layered device or component to be laser processed is machined prior to assembly. For example FIG. 9 A illustrates a microfluidic device manufactured by laser engraving the substrate (63) prior to bonding the top layer (64). In another example FIG. 9 B illustrates a microfluidic structure formed by cutting entirely through a layer (66) before sealing with substrates (65,67) above and below.

In another preferred embodiment of the invention the device or component to be laser processed is machined after assembly into a multi-layered component or device. For example, FIG. 10 illustrates channel formation in a microfluidic device by laser machining. In this example the top layer (69) is significantly transparent to the laser beam (68) and one or more of the lower layers (70) absorb significant amounts of the laser energy enabling the formation of internal structures such as vias, chambers and channels (71). Such a technique is also particularly useful for removing swarf, debris, and cut-out areas by using one or more of the layers bonded to the machined layer as a sacrificial layer and removing it after the machining process. Alternatively the machining process may bond the machined layer to its adjacent layers, or improve the bonding of such layers, by localised melting and reflow induced by the laser machining process around the machined areas.

In another embodiment of the invention the device or component may incorporate layers that act as masking components to guide the radiation onto specific locations. This approach allows the use of larger laser beams to create smaller structures than normally achievable with the full beam exposure. The use of larger beam lasers and laser curtains may also be used to increase the throughput of the machining process by enabling parallel machining from the same laser beam. Such a method also offers the advantage of decreasing the alignment requirements for the laser system by using a mask to provide tight tolerances. Such a masking system may also provide greater spatial resolution in a similar manner to traditional lithographic systems. Furthermore, such a masking system may also provide manufacturing advantages if the mask is part of the manufactured component by simplifying alignment between features on a single device and between each manufactured part. Furthermore the masking material may be used to (a) improve the thermal heat affected zone on the sample by conducting some of the heat away from the structured area, (b) protect the substrates surface from debris, and/or (c) support the machined structures during processing. FIG. 11 depicts a mask (71) limiting the exposure of a material (72) to a relatively large laser beam or curtain (73).

In another embodiment of the invention the device or component may incorporate layers that use optical components, such as lenses, prisms or other refractive or diffractive features, to focus and/or redirect the radiation onto specific locations. This method also offers the advantage of decreasing the alignment requirements for the laser system by using the optical components to provide the tight tolerances required. Such optical components may provide greater spatial resolution by focussing the radiation. In addition, such optical components may also provide manufacturing advantages by having the optical components as a part of the manufactured component and thus simplifying alignment between features on a single device and between each manufactured part.

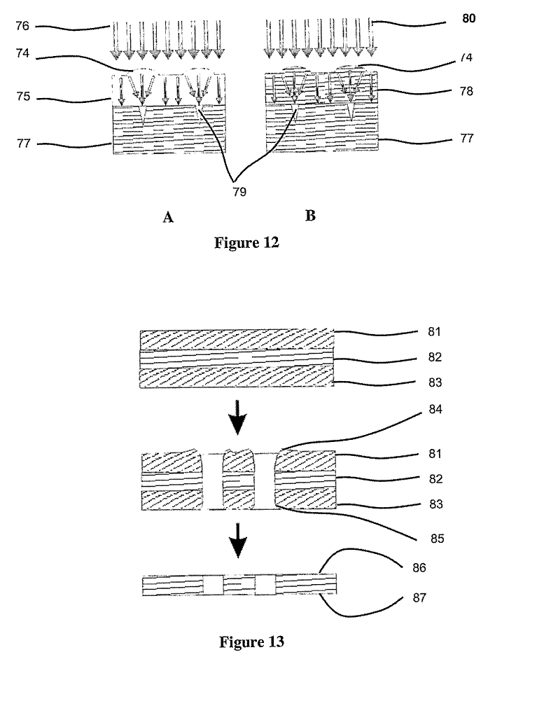

FIG. 12 illustrates an example of optical components integrated onto a part to focus the laser radiation. The example in FIG. 12 A illustrates lenses (74) moulded onto the surface of a material (75) that is transparent to the laser beam, the focused radiation provides greater localised intensities that process a second material (77) at a higher rate, or is above the ablation threshold, in comparison to the unfocussed radiation. Similarly the example of FIG. 12 B shows a material (78) that is semi transparent to laser radiation (80) and at the high intensity points where the radiation is focused localised machining occurs (79).

In some embodiments of the invention, the mutilayer parts have layers removed after the laser machining process, or after parts of the manufacturing process. Extra layers may be used during the machining process for various reasons, for example to protect the surface from debris, act as a thermal conductor to minimise the heat affected zone on the machined substrate, and support cut out, or free standing, structures as outline in US PCT/AU2007/000061. The layers may also be used during the machining process to focus or mask a beam, provide heat conduction, or allow a secondary machining process to occur.

The example in FIG. 13 illustrates protective layers being used to improve the laser machining process. In this example the substrate (82) has two protective layers (81, 83), during the machining process all three materials are cut entirely through. Many machining processes cause deformation around the cut at the top (84) and bottom surfaces (85). By removing the outer sacrificial layers (81, 83) the inner substrate (82) is left with relatively clean surfaces (86, 87) and allows for reduced thermal damage in the surrounding area.

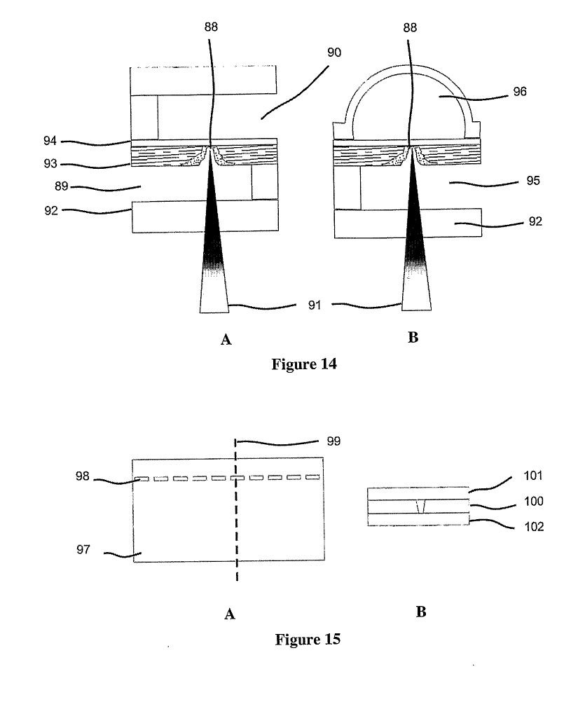

In one embodiment of the invention the selectively machined layer is used to weaken the surrounding structure to form a burst valve. These burst valves can be made by partially machining through a layer of a multilayer device or entirely machining through one layer and leaving a thin adjacent layer that may rupture under pressure. A layer can be selectively machined by using an adjacent transparent, heat conductive or reflective layer. FIG. 14 illustrates burst valves in a microfluidic device fabricated by machining entirely through a layer with transparent adjacent layers. FIG. 14 A shows an example of the formation of a burst valve (88) between two adjacent channels (89, 90), by laser machining (91) through substrate (92) transparent to the laser radiation and etching an inner layer (93) leaving only a thin non-absorbing layer (94) of material that can be burst under pressure. FIG. 14 B illustrates a similar structure except that the burst valve is formed between the channel (95) and thermoformed liquid reservoir (96). For this liquid storage example, the thin non-machined layer adjacent to, and in contact with, the machined layer may for example have improved barrier and chemical compatibility properties in comparison to the laser-absorbing layer.

In one embodiment of the invention the selectively machined layer is used to weaken the surrounding structure to form a tearing guide. For example, FIG. 15 illustrates a machined substrate (97) that provides a tearing line (98) for packaging. Where some of the structural layers are machined to provide a controlled tearing line for the user but still maintain the barrier properties of the packaging. The dotted line (99) down the centre of FIG. 15 A represents the cross section line for the image shown in FIG. 15 B. FIG. 15 B illustrates that the inner substrate (100) is perforated whilst the outer layers (101, 102) remain intact.

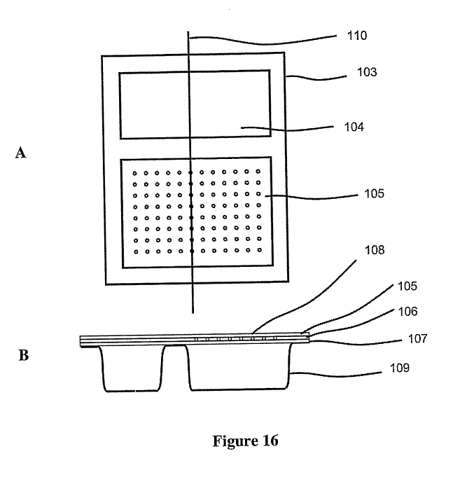

In one embodiment of the invention the selectively machined layer is used to perforate selected layers of a multi-layer material to alter the barrier properties of the device. This technique provides the added advantage of allowing spatial control of the barrier properties on a multi-layer device such as packaging using the same materials and fabrication process for the entire package. In the following example, shown in FIG. 16, a two pack thermoformed tray (103) uses the same sealing multilayer laminate but provides different barrier properties to each tray (104,105) from the machining process. The dotted line (110) down the centre of FIG. 16 A represents the cross section line for the image shown in FIG. 16 B. In this example the thermoformed tray (109) is sealed by the three laminate layers (105, 106, 107), and the central layer (106) is perforated to alter the barrier properties to one of the tray containers.

1-140. (canceled)

141. A method for manufacturing at least part of a single- or multilayered device comprising use of at least one laser to alter at least one layer of said part during the manufacturing process.

142. A method according to claim 141 wherein more than one wavelength is used.

143. A method according to claim 141 comprising use of a plurality of laser beams.

144. A method according to claim 141 comprising the use of an additive in a layer to alter the effect of a laser beam on that or another layer.

145. A method according to claim 141 comprising the use of a layer with an absorption and/or reflection characteristic to influence the effect of the laser.

146. A method according to claim 141 comprising the use of a masking component between the laser source and a layer to limit or alter exposure to the laser beam on an area of the layer.

147. A method according to claim 141 comprising the use of a masking component between the laser source and the substrate to limit or alter exposure to the laser beam on a portion of the substrate.

148. A method according to claim 141 wherein a layer is removed during or after the manufacturing process.

149. A method according to claim 141 wherein the process utilizes an at least semi-continuous web.

150. A method according to claim 141 wherein the device is a microfluidic device.

151. An apparatus for manufacturing at least part of a single- or multilayered device comprising at least one laser source to produce a laser beam to alter at least one layer of said part during the manufacturing process.

152. An apparatus according to claim 151 wherein the device is a microfluidic device.

153. A single- or multilayered device or part thereof manufactured according to the method of claim 141, or manufactured using the apparatus of claim 151.

154. A device or part thereof according to claim 153 wherein the device is a microfluidic device.

155. A method for laser machining a single- or multilayered material, the method comprising the step of irradiating one or more layers of the material, wherein the one or more layers of the material have an optical component or a heat conduction characteristic that allows for interaction with the laser.

156. A method for laser machining a single- or multilayered material, the method comprising the steps masking a component of the material, and laser irradiating the masked component, wherein the mask forms part of the manufactured component during the laser machining process.

157. A method for laser machining a single- or multilayered material, the method comprising the steps of modifying a surface property or a bulk property of the material to alter the thermal conductivity or absorption or optical characteristic, and laser irradiating the material.
